(19) United States
(12) Patent Application Publication
(10) Pub. No.: US 2009/0143782 A1
(43) Pub. Date: Jun. 4, 2009

(54) MICROFRACTURE SURGERY APPARATUS AND METHOD

(76) Inventor: JAMES H. LEVI, TUCSON, AZ (US)

Correspondence Address: DALE F. REGELMAN, QUARLES & BRADY, LLP, ONE SOUTH CHURCH AVENUE AVE, STE. 1700, TUCSON, AZ, 85701-1621, US

(21) Appl. No.: 12/275,104

(22) Filed: Nov. 20, 2008

Related U.S. Application Data
(60) Provisional application No. 60/991,303, filed on Nov. 30, 2007.

(51) Int. Cl. A61B 17/00; A61B 17/32

(52) U.S. Cl. 606/79; 606/167

(57) ABSTRACT

Apparatus and method for microfracture surgery to create apertures in a bone tissue surface comprising uniform depth by controlling an insertion length. Verification of insertion length may be accomplished through the use of a color-coded kit wherein each microfracture surgery apparatus comprises a color-coded handle associated with an insertion length and offset angle.

### CROSS-REFERENCE TO RELATED APPLICATION

This application incorporates by reference and claims priority to U.S. Provisional Application Ser. No. 60/991,303, which was filed on Nov. 30, 2007.

### FIELD OF THE INVENTION T

he invention relates to a microfracture surgery apparatus and a method for facilitating the growth of new cartilage. In certain embodiments, the invention comprises a kit comprising a plurality of microfracture surgical devices.

### BACKGROUND OF THE INVENTION

Microfracture surgery is an orthopedic surgical technique that can help restore knee cartilage by creating tiny fractures in the adjacent bones, causing new cartilage to develop. It can be used to treat both degenerative knee problems as well as cartilage injuries. The surgery is performed through an arthroscopy. The surgeon first removes any damaged cartilage. Tiny fractures are then created in the adjacent bones through the use of an awl. Blood and bone marrow (which contains stem cells) seep out of the fractures, creating a blood clot that releases cartilage-building cells. The microfractures are treated as an injury by the body, which is why the surgery results in new, replacement cartilage.

### BRIEF DESCRIPTION OF THE DRAWINGS

The invention will be better understood from a reading of the following detailed description taken in conjunction with the drawings in which like reference designators are used to designate like elements, and in which:

FIG. 1A is a perspective view of a prior art surgical device;

FIG. 1B is a perspective view of Applicant's microfracture surgery apparatus;

FIG. 2A shows a second embodiment for the handle portion of the microfracture surgery apparatus of FIG. 1B;

FIG. 2B shows a third embodiment for the handle portion of the microfracture surgery apparatus of FIG. 1B;

FIG. 2C shows a fourth embodiment for the handle portion of the microfracture surgery apparatus of FIG. 1B;

FIG. 3 is a side view of extension member 120 and offset extension member 130;

FIG. 4 is a side view of offset extension member 130 and insertion portion 130;

FIG. 5A shows a side view of one embodiment of the tip portion of Applicant's microfracture surgery apparatus;

FIG. 5B shows a top view of the tip portion of FIG. 5A.

### DETAILED DESCRIPTION OF THE PREFERRED EMBODIMENTS

This invention is described in preferred embodiments in the following description with reference to the Figures, in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of the invention may be combined in any suitable manner in one or more embodiments. In the following description, numerous specific details are recited to provide a thorough understanding of embodiments of the invention. One skilled in the relevant art will recognize, however, that the invention may be practiced without one or more of the specific details, or with other methods, components, materials, and so forth. In other instances, well-known structures, materials, or operations are not shown or described in detail to avoid obscuring aspects of the invention.

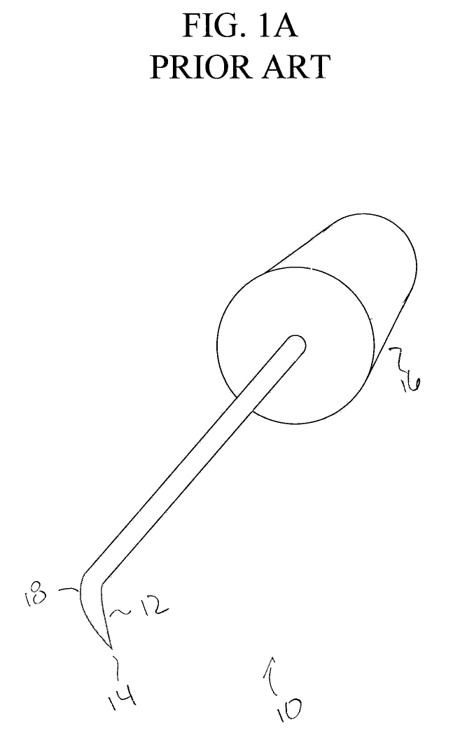

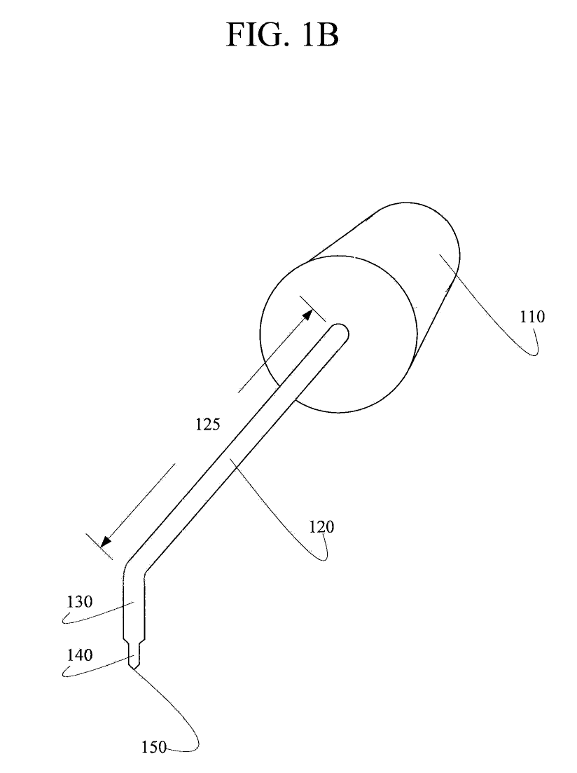

As those skilled in the art will appreciate, injured joint surface cartilage does not heal. Using microfracture surgery techniques, a surgeon first denudes the bone surface by removing any injured bone surface cartilage. Holes are then formed in the denuded bone surface. FIG. 1A illustrates a prior art device 10, sometimes referred to as a chondro pick, used to form holes in a bone surface during microfracture surgery. Tip 14 is placed against a bone surface, and insertion portion 12 is advanced into the bone. A mallet may be used to tap handle 16 thereby advancing tip 14 inwardly into bone tissue.

Insertion portion 12 of prior art chondro pick 10 comprises an awl-type structure wherein insertion portion 12 tapers continuously and smoothly from a first diameter of about 0.5 inches at point 18 to a sharp point. As those skilled in the art will appreciate, the deeper the penetration of insertion portion 12 into bone tissue, the greater the injury to that hard tissue. Using prior art device 10, it is difficult to advance insertion portion 12 a consistent distance into bone tissue. If too little force is applied, then insertion portion 12 is not advanced the desired distance into hard tissue. On the other hand, if too much force is applied, then insertion portion 12 can be advanced beyond a nominal and optimal distance into hard tissue.

Referring now to FIG. 1B, Applicant's microfracture surgery apparatus 100 comprises handle 110, extension member 120, offset extension member 130, and insertion portion 140. In certain embodiments, extension member 120 is between about 4 inches and about 10 inches in length 125. In various embodiments, Applicant's microfracture surgery apparatus 100 can be used in surgical procedures involving various joints disposed in animals, including humans. As those skilled in the art will appreciate, by “joint,” Applicant means an areas where two or more bones meet, wherein most joints are mobile allowing the bones to move relative to one another.

Such joints include, for example and without limitation, knee joints, hip joints, elbow joints, shoulder joints, and the like. As a result, optimal dimensions for handle 110 and extension member 120 may vary as a function of the anatomy of the joint being repaired.

In certain embodiments, extension member 120, offset extension member 130, and insertion portion 140 are formed from stainless steel. As those skilled in the art will appreciate, stainless steel is defined as an iron-carbon alloy with a minimum of 10.5% chromium content. In certain embodiments, extension member 120, offset extension member 130, and insertion portion 140 are formed from Type 630 stainless steel, better known as 17-4; 17% chromium, 4% nickel. In certain embodiments, extension member 120, offset extension member 130, and insertion portion 140 are heat-treated, and comprise a Rockwell Hardness between about 40 to 44.

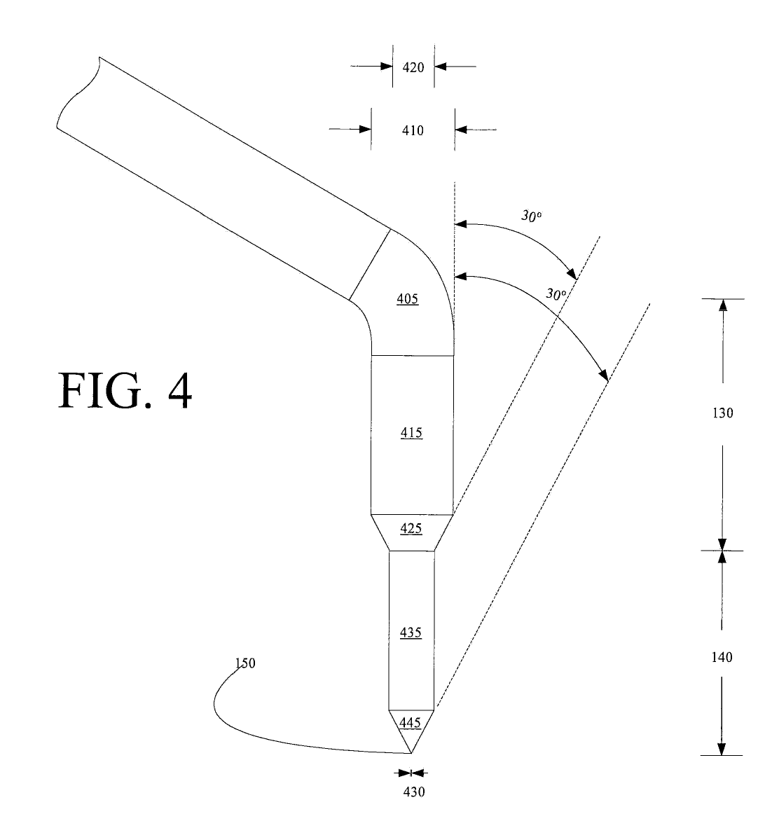

Applicant's microfracture surgery apparatus 100 tapers twice in two discontinuous locations, first from cylindrical member 415 (FIG. 4) to a first end of cylindrical member 435 (FIG. 4) via truncated conical member 425 (FIG. 4), and then from a second end of cylindrical member 435 to tip portion. Cylindrical member 435 comprises a constant diameter from a first end to a second end. Applicant has found that use of a wide range of applied forces to handle 110 causes the entire length of insertion portion 140 into hard tissue. As a result, a uniform depth and diameter of holes formed in bone tissue can be achieved when using Applicant's microfracture surgery apparatus 100. In marked contrast, use of tapering prior art devices results in holes of varying depth, and also varying diameter. The use of applied force may be caused by tapping handle 110 with a hard instrument like a mallet or a machine-operated structure.

In the illustrated embodiment of FIG. 1B, handle 110 is shown comprising a cylindrical shape. In certain embodiments, handle 110 is formed from a rigid material, such as an engineering plastic, metal, and combinations thereof. By “engineering plastic,” Applicant means a polymeric material comprising a tensile modulus of about 500,000 psi or greater, and/or a flexural modulus of about 500,000 psi or greater. In certain embodiments, handle 110 is formed from aluminum.

Such polymeric materials include, without limitation, one or more polyamides, one or more polyimides, one or more polyetheretherketones, one or more cured epoxy resins, and the like. As those skilled in the art will appreciate, a handle formed using an engineering plastic will have a weight of about 0.25 to about 0.50 times the weight of a metal handle. In certain embodiments, handle 110 is formed from a cellular material having a density of about one half that a handle formed using a comparable non-cellular metal or plastic.

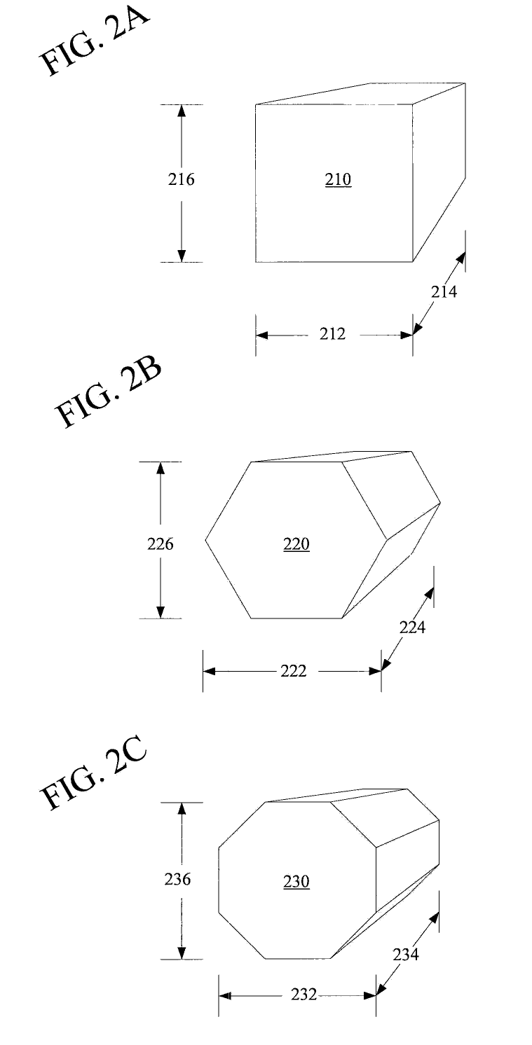

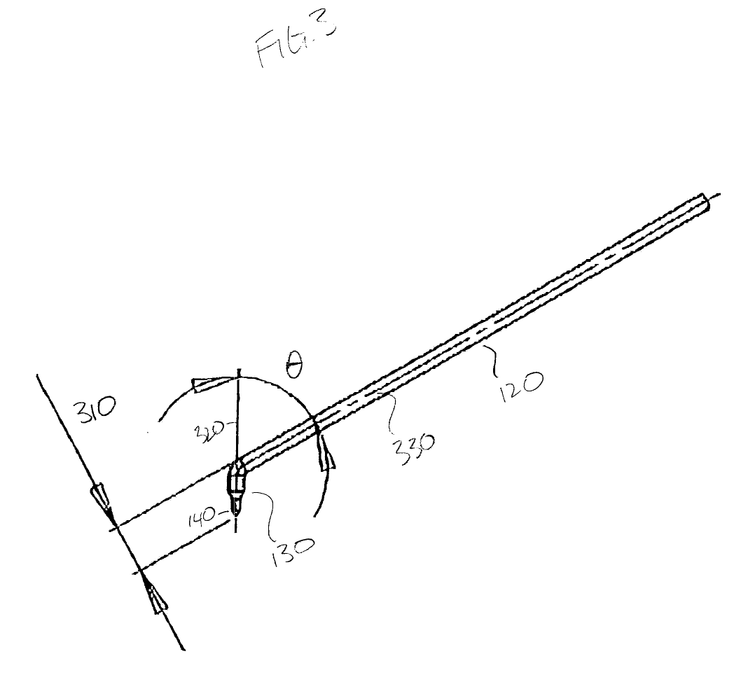

Referring now to FIGS. 2A, 2B, and 2C, in other embodiments handle 110 comprises a parallelepiped such as handle 210. In certain embodiments, handle 210 comprises a square cross-section, wherein width 212 equals height 216. In other embodiments, handle 210 comprises a rectangular cross-section, wherein width 212 does not equal height 216.

In certain embodiments, width 212 is between about 0.5 inches and about 2 inches. In certain embodiments, height 216 is between about 0.25 inches and about 1 inch. Handle 210 further comprises a length 214, wherein length 214 is between about 4 inches and about 8 inches.

Referring now to FIG. 2B, in other embodiments handle 110 comprises a hexagonal cross-section, such as handle 220. Handle 220 comprises width 222, height 226, and length 224. In certain embodiments, width 222 is between about 0.5 inches and about 2 inches. In certain embodiments, height 226 is between about 0.25 inches and about 1 inch. In certain embodiments, length 224 is between about 4 inches and about 8 inches.

Referring now to FIG. 2C, in other embodiments handle 110 comprises an octagonal cross-section, such as handle 230. Handle 230 comprises width 232, height 236, and length 234. In certain embodiments, width 232 is between about 0.5 inches and about 2 inches. In certain embodiments, height 236 is between about 0.25 inches and about 1 inch. In certain embodiments, length 234 is between about 4 inches and about 8 inches.

Referring now to FIG. 3, a centerline 320 of offset extension member 130 and a centerline 330 of extension member 120 define an offset angle Θ. In certain embodiments, offset angle Θ equals 0 degrees. In certain embodiments, offset angle Θ is about 15 degrees. In certain embodiments, offset angle Θ is about 30 degrees. In certain embodiments, offset angle Θ is about 45 degrees. In certain embodiments, offset angle Θ is about 60 degrees. In certain embodiments, offset angle Θ is about 90 degrees.

Offset extension member 130 in combination with insertion portion 140 10 comprises an length 310. In certain embodiments, length 310 is about 0.25 inches. In certain embodiments, length 310 is about 0.50 inches.

In certain embodiments, handle 110 is color-coded with various markings to separately indicate the different offset angles Θ and lengths 310. The following example is presented to further illustrate to persons skilled in the art how to make and use the invention. This example is not intended as a limitation, however, upon the scope of the invention.

### EXAMPLE

In certain embodiments handle 110 comprises a first color-coded marking to indicate a first embodiment of microfracture surgery apparatus 100 wherein length 310 is 0.25 inches, and angle Θ is 30 degrees, wherein a first insertion length marking is identified from a group of varying colors, shades, and other graphics and a first offset angle marking is identified from a group of varying colors, shades, and other graphics.

In certain embodiments handle 110 comprises a second marking to indicate a second embodiment of microfracture surgery apparatus 100 wherein length 310 is 0.50 inches and angle Θ is 15 degrees, wherein a second length marking is identified from a group of varying colors, shades, and other graphics, wherein second insertion length marking differs from said first insertion length marking and a second offset angle marking is identified from a group of varying colors, shades, and other graphics, wherein second offset angle marking differs from said first offset angle marking.

In certain embodiments handle 110 comprises a third marking to indicate a third embodiment of microfracture surgery apparatus 100 wherein length 310 is 0.25 inches and angle Θ is 90 degrees, wherein a third offset angle marking is identified from a group of varying colors, shades, and other graphics, and wherein the third offset angle marking differs from the first and second offset angle markings and a third insertion length marking is identified from a group of varying colors, shades, and other graphics, wherein third insertion length marking differs from the first and second insertion length markings.

In certain embodiments handle 110 comprises a fourth marking to indicate a fourth embodiment of microfracture surgery apparatus 100 wherein length 310 is 0.50 inches and angle Θ is 60 degrees, wherein a fourth offset angle marking is identified from a group of varying colors, shades, and other graphics, and wherein the fourth offset angle marking differs from the first, second and third offset angle markings and a fourth insertion length marking is identified from a group of varying colors, shades, and other graphics, wherein fourth insertion length marking differs from the first, second, and third insertion length markings.

Referring now to FIGS. 1B and 4, offset extension member 130 comprises wedged-shaped member 405, cylindrical member 415, and truncated conical member 425. Wedged-shaped member 405 and cylindrical member 415 comprise a diameter 410. In certain embodiments, diameter 410 is between about 0.06 inches to about 0.20 inches. In certain embodiments, diameter 410 is about 0.10 inches. Truncated conical member 425 tapers from diameter 410 to diameter 420. In certain embodiments, diameter 420 is between about 0.04 inches to about 0.125 inches.

Insertion portion 140 comprises cylindrical member 435 and conical member 445. Cylindrical member 435 comprises diameter 420. Conical member 445 tapers from diameter 420 to diameter 430 at tip portion 150, wherein diameter 430 is between about 0 inches to about 0.010 inches.

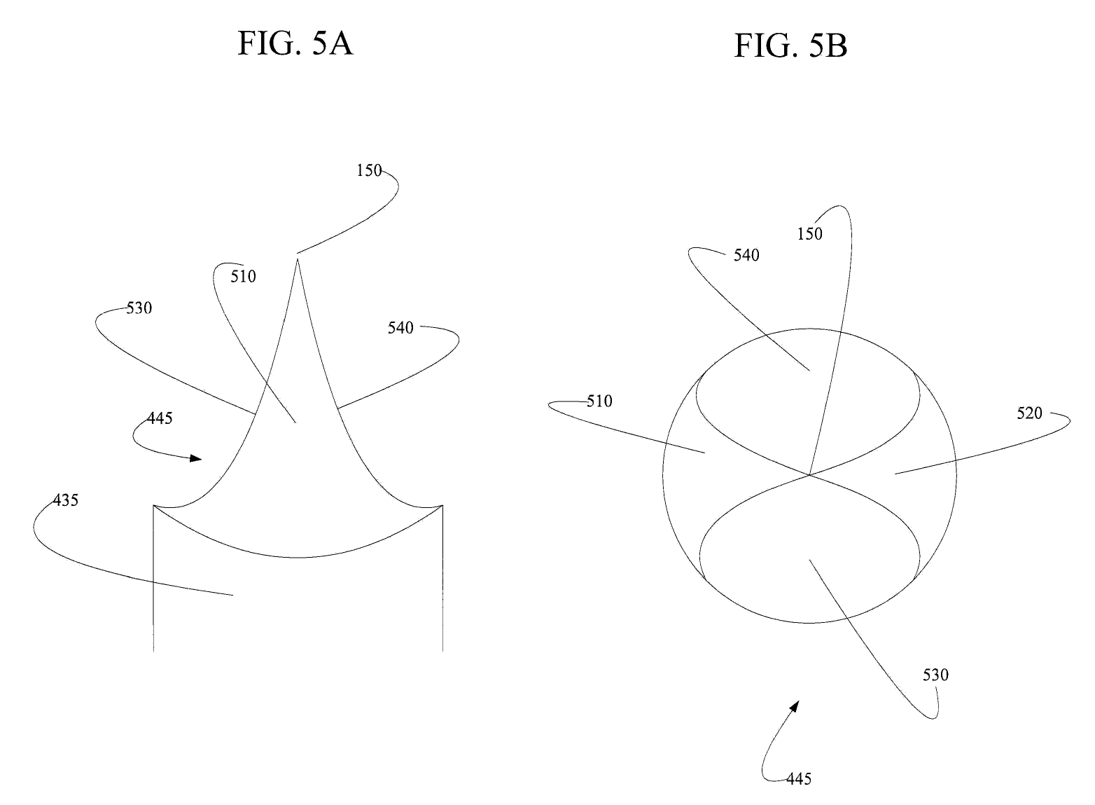

In certain embodiments conical member 445 uniformly tapers to tip portion 150 as shown in FIG. 4. In other embodiments, conical member 445 comprises a more complex shape. For example in the illustrated embodiment of FIGS. 5A and 5B, conical member 445 comprises 4 surfaces, namely surfaces 510, 520, 530, and 540. FIG. 5A shows a side view of this embodiment of conical member 445, and FIG. 5B shows a top view. Opposing surfaces 530 and 540 have been “flattened” such that conical member 445 comprises a “chisel-like” shape.

Cylindrical member 435, and conical member 445, in combination, comprise insertion portion 140. In certain embodiments, insertion portion 140 comprises a length between about 0.20 inches and about 0.30 inches. In certain embodiments, insertion portion 140 comprises a length of about 0.24 inches.

While the preferred embodiments of the present invention have been illustrated in detail, it should be apparent that modifications and adaptations to those embodiments may occur to one skilled in the art without departing from the scope of the present invention.

What is claimed is:

1. A microfracture surgery apparatus, comprising:
a handle;
an extension member extending outwardly from said handle;
an offset extension member connected to said extension member, wherein said offset extension member and said extension member define an offset angle;
wherein said offset extension member comprises:
a wedged-shaped member attached to and extending outwardly from said extension member and comprising a first diameter;
a first cylindrical member comprising a first end, a second end, and said first diameter, wherein said first end is attached to said wedged shaped member;
a truncated conical member having a first end comprising said first diameter and a second end comprising a second diameter, wherein said first end is attached to said second end of said first cylindrical member;
an insertion portion comprising a second diameter, wherein said first diameter is greater than said second diameter, wherein said insertion portion comprises:
a second cylindrical member comprising a first end, a second end, and said second diameter, wherein said first end of said second cylindrical member is attached to said second end of said truncated conical member;
a conical member connected to said second end of said second cylindrical member, wherein said second cylindrical member tapers to a tip portion comprising a third diameter; and
wherein when said tip portion is placed onto a bone surface and a force is applied to said handle, said apparatus penetrates into said bone a distance no greater than said insertion length.

2. The apparatus of claim 1, wherein said second conical member comprises four interconnected surfaces, wherein a pair of opposing surfaces are flattened to define a chisel-like shape.

3. The apparatus of claim 1, wherein said insertion length is selected from the group consisting of 0.25 inches and 0.50 inches.

4. The apparatus of claim 1, wherein said offset angle is selected from the group consisting of 0, 15, 30, 45, 60 and 90 degrees.

5. The apparatus of claim 1, wherein said first diameter is about two times said second diameter.

6. The apparatus of claim 1, wherein said first diameter is about five times said second diameter.

7. The apparatus of claim 1, wherein said the second diameter is about four times said third diameter.

8. The apparatus of claim 1, wherein said second diameter is about ten times said third diameter.

9. A method of facilitating tissue growth of new cartilage by utilizing a microfracture surgery apparatus, comprising the steps of:
supplying a microfracture surgery apparatus, comprising a handle, an extension member connected to said handle, an offset extension member connected to said extension member, wherein said offset extension member and said extension member define an offset angle, wherein said offset extension member comprises a wedged-shaped member, a first cylindrical member wherein said wedged-shaped member and said first cylindrical member comprise a first diameter, a first truncated conical member, an insertion portion connected to said offset extension -member and comprising an insertion length and a second diameter, wherein said first diameter is greater than said second diameter, and wherein said insertion portion comprises, a second cylindrical member, a second conical member comprising said second diameter, and tapering to a tip portion having a third diameter;
identifying a degenerative bone tissue surface;
denuding said bone surface;
removing from said bone surface any calcified bone cartilage;
positioning said tip portion of apparatus adjacent to said bone surface;
applying a force to said handle of said apparatus;
penetrating into said bone surface by said insertion portion a distance no greater than said insertion length;

10. The method according to claim 7, further comprising selecting an insertion length from the group consisting of 0.25 inches and 0.50 inches.

11. The method according to claim 7, further comprising the step of selecting said offset angle from the group consisting of 0, 15, 30, 45, 60 and 90 degrees.

12. The method of claim 7, wherein:
said first diameter is about 0.06 inches to about 0.20 inches;
said second diameter is about 0.04 inches to about 0.10 inches; and said third diameter is less than or equal to 0.010 inches.

13. A surgical kit for microfracture surgery comprising:
a plurality of microfracture surgical assemblies, wherein said assembly comprises:
a handle attached to an extension member;
an offset extension member interconnected with said extension member forming an offset angle to facilitate penetrating a degenerative bone surface,
wherein said offset extension member comprises:
a wedged-shaped member;
a first cylindrical member having a first diameter;
a first truncated conical member configured to taper from said first diameter;
an insertion portion connected to said first truncated conical member forming an insertion length configured to create a uniform depth aperture into said bone surface having a second diameter, wherein said first diameter is greater than said second diameter,
wherein said insertion portion comprises:
a second cylindrical member configured to connect to a second conical member that tapers from said second diameter to a tip portion having a third diameter configured to be attached to a distal end of said insertion portion.

14. The surgical kit of claim 12, wherein said second conical member comprises four interconnected surfaces, wherein a pair of opposing surfaces are flattened to define a chisel-like shape.

15. The surgical kit of claim 12, wherein said first diameter is about two times said second diameter.

16. The surgical kit of claim 12, wherein said first diameter is about five times said second diameter.

17. The surgical kit of claim 12, wherein said the second diameter is about four times said third diameter.

18. The surgical kit of claim 12, wherein said second diameter is about ten times said third diameter.

19. The surgical kit of claim 12, wherein each assembly comprises a color-coded handle indicating an insertion length, wherein said insertion length is selected from the group consisting of 0.25 inches and 0.50 inches.

20. The surgical kit of claim 18, wherein each assembly comprises a color-coded handle that further indicates an offset angle, wherein that offset angle is selected from the group consisting of 0, 15, 30, 45, 60 or 90 degrees.
